(19) United States
(12) Patent Application Publication
(10) Pub. No.: US 2009/0118737 A1
(43) Pub. Date: May 7, 2009

(54) COUNTER ANGLE FOR IMPLANTOLOGY OR SURGERY COMPRISING REMOVABLE SUB-ASSEMBLIES AND IN PARTICULAR AN INTERMEDIATE POSITIONING SUPPORT FOR THE HEAD CARTRIDGE

(75) Inventors: Jacques Pernot, Vieilley (FR); Jean Claude Boinot, Rigney (FR)

Correspondence Address: EGBERT LAW OFFICES, 412 MAIN STREET, 7TH FLOOR, HOUSTON, TX, 77002, US

(73) Assignee: MICRO MEGA INTERNATIONAL MANUFACTURES (Societe Anonyme), Besancon (FR)

(21) Appl. No.: 12/302,284

(22) PCT Filed: Aug. 3, 2007
(86) PCT No.: PCT/FR07/51777

§ 371 Date: Nov. 24, 2008

(30) Foreign Application Priority Data
Aug. 7, 2006 (FR) 06/07177

(51) Int. Cl. A61B 17/58

(52) U.S. Cl. 606/99

(57) ABSTRACT

The invention relates to a contra-angle for implantology or surgery with a head for holding and rotating an instrument about its longitudinal axis, and a handpiece containing, in a non-limiting manner, a connection providing the connection with an external motor drive, and a reducing assembly with an output gear. The contra-angle is formed of removable subassemblies, and principally of a subassembly called the intermediate support, including an input gear meshing with the output gear of the reducing assembly and an end gear mounted on the same rotating shaft and inside a hollow cylindrical body. The intermediate support is mounted so as to slide freely along an axis inside a central part of a head body and enabling positioning in the head and rotational driving of a removable head cartridge, when all the subassemblies are mounted together to form the contra-angle.

The present invention relates to a contra-angle for implantology or surgery comprising removable subassemblies.

Contra-angles designed for implantology or surgery are subjected to heavy loads, due to mechanical stresses (couple of around 70 Ncm and to environmental constraints such as blood, saliva, debris, bone, etc.).

Between each patient, hygiene rules therefore require thorough cleaning before sterilization. To obtain an effective level of cleaning, it is essential that the main subassemblies of these types of contra-angle are properly removable and remountable, as easily as possible, without tools, without particular effort, without much training, but with high efficiency and reliability, whence the object of the present invention.

This object is attained by a contra-angle designed for implantology or surgery with a head for holding and rotating an instrument about its longitudinal axis, and a handpiece containing, in a nonlimiting manner, a connection providing the connection with an external motor drive, and a reducing assembly with an output gear, characterized in that the contra-angle is formed of removable subassemblies including two coaxial subassemblies of a removable handpiece and a subassembly called the intermediate support comprising an input gear meshing with the output gear of the reducing assembly and an end gear mounted on the same rotating shaft and inside a hollow cylindrical body, said intermediate support being mounted so as to slide freely along an axis inside a central part of a head body and enabling positioning in the head and rotational driving of a removable head cartridge, when all the subassemblies are mounted together to form the contra-angle, the intermediate support comprising a means for indexing the head body in relation to the handpiece.

According to a variant, the intermediate support is positioned obliquely in the head body, in a direction forming an angle with the direction of the instrument.

The removable subassemblies of the handpiece preferably comprise means for quick indexing in relation to each other.

According to a first embodiment of the axial positioning of the cartridge by the intermediate support, one or more flat and axially directed returns of an end cut-out of a cylindrical cover of the intermediate support are applied on flat bearing surfaces provided in the head cartridge and perpendicular to the axis of rotation of the head, a preloaded spring washer being provided between the underside of the head cartridge and a bottom bearing plane of the head body, and limiting the residual play between the cartridge and the head body.

The intermediate support being then positioned in the stop position on a shoulder inside the head body and perpendicular to the axis of the support, while the cartridge is positioned bearing completely on a shoulder of the head body perpendicular to the axis of rotation of the tool.

According to another embodiment of the axial positioning, the intermediate support is equipped with a telescopic locking system formed of a sliding lock in the form of a cylindrical sleeve sliding inside a coaxial bushing aligned with the outer body of the support, the sliding lock comprising a projecting internal stop capable of compressing a spring held by a bushing base, and comprising an outwardly projecting lug sliding in a guiding slot of the bushing parallel to the longitudinal axis of the support.

In all cases, the body of the support also comprises an indexing means, preferably an extension in the form of a tongue projecting toward the handpiece of the contra-angle, parallel to the axis, passing beyond the input gear and capable of being housed, when the support is positioned in the head body, in an opening in a cylindrical part of the head body and forming a bend in relation to the central part of the head body.

Moreover, the handpiece is formed by putting together two subassemblies, including an inner subassembly that supports a reducing assembly and slides freely within an outer subassembly, the inner subassembly comprising a cylindrical overhang passing beyond the output gear of the reducing assembly and comprising a slot and into which the tongue of the intermediate support fits.

In all cases, the contra-angle is removable in two parts, head and handpiece, each capable of being removed as subassemblies. Thus the head-handpiece connection is the first to be removed and the last to be remounted. It must comprise a reliable indexing means, to ensure force transmission, provided on the intermediate support.

In addition, in all cases the handpiece-head connection is preferably locked by a central, axially displaceable casing having a withdrawn position, preferably but not limitingly retracted into the handpiece, enabling all the subassemblies forming the contra-angle to be disconnected, and a working position joining the handpiece to the head body. Equivalently, the casing may conversely be provided on the head body and have a retracted position in the head body.

The invention will be understood better with the help of the description below, provided with reference to the following appended figures:

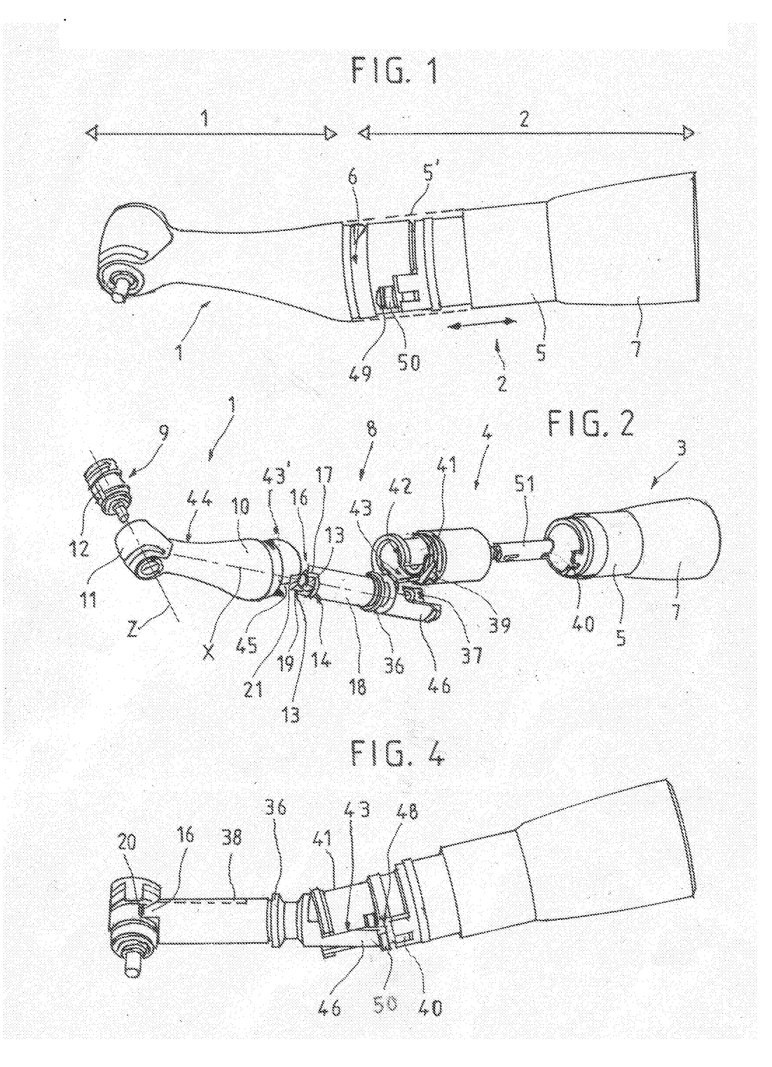

FIG. 1: general view of a contra-angle according to the invention;

FIG. 2: exploded view of a contra-angle according to the invention and according to a first mode of axial positioning;

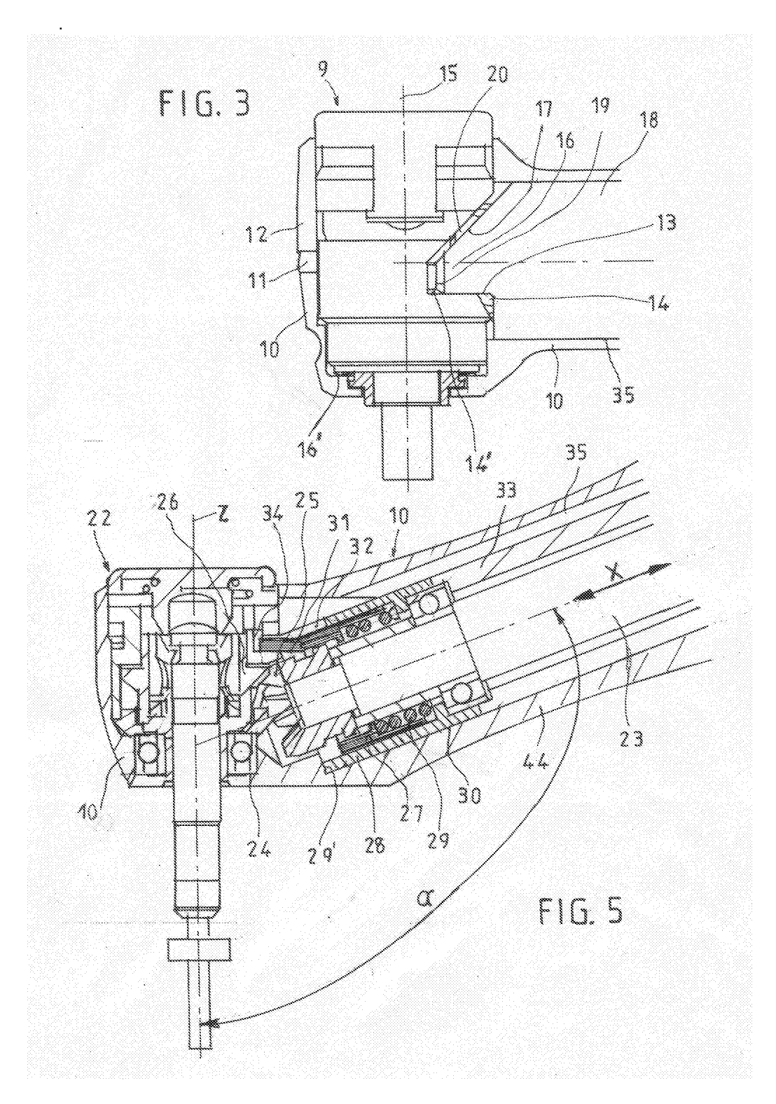

FIG. 3: partial cross-sectional view of the contra-angle of FIG. 2;

FIG. 4: general view showing the intermediate support in position, the head body not being shown; and

FIG. 5: partial cross-sectional view of a contra-angle according to the invention according to another mode of axial positioning.

A contra-angle according to the invention is composed of a head (1) mounted, so as to be easily removable and remountable, on a handpiece (2) formed of two coaxial and separable subassemblies (3) and (4).

In the use configuration (not shown), a central, axially displaceable casing (5) joins the handpiece (2) and the head body (10) bearing the head (1), for example by being screwed onto an external thread (6) of the head (1).

In the nonlimiting embodiment shown in the figures, the casing has a working position (shown with the dotted line (5′)) in which it is joined to the head and a withdrawn position, preferably retractable, inside a cylindrical or approximately conical deflector (7) of the handpiece (2), this position being represented in the figures and in particular in FIG. 1, for example after unscrewing and then axial displacement.

Reference will now be made to FIG. 2, which shows the contra-angle according to the invention separated into subassemblies with:
the two handpiece subassemblies, an outer subassembly (3) and an inner subassembly (4) respectively;
an intermediate support (8); and
a head (1) with a head cartridge (9) and a head body (10).

The peculiarity of the device according to the invention lies mainly in the concept of retaining the head cartridge (9) in the head body (10) by means of an action by the intermediate support (8), for example, but not limited thereto, in an oblique position relative to the direction of the head body (10).

The principle of the removable head cartridge is already known per se. It is a subassembly comprising mainly means for holding an instrument and for rotating this instrument about its longitudinal axis. The rotational movement is transferred to gears of the removable cartridge after joining and locking this cartridge into the head body (10).

In the case described, a pin (11) joined to the head body (10) positions the cartridge and prevents it from rotating, which cartridge is provided with an axial groove (12) cooperating with said transverse pin. In another embodiment (not shown), a lug joined to the cartridge is able to cooperate with a groove made in the head body. The head cartridge (9) slides freely into the head body (10).

The axial positioning of the cartridge (9) and the rotational drive are ensured by introducing the intermediate support (8) into the head body (10).

FIGS. 2 to 4 show an embodiment of axial positioning of said cartridge by the intermediate support (8).

One or more flat (and axially directed) returns (13) of an end cut-out (14) of a cylindrical cover (18) of the intermediate support (8) are applied to planar supports (14′) provided in the head cartridge and perpendicular to the axis of rotation (15) of the head.

A preloaded spring washer (16′) provided between the underside of the head cartridge and a bottom bearing plane of the head body limits the residual play between the cartridge (9) and the head body (10).

The flat returns (13) furthermore constitute the undersides of a nose-shaped overhang (16) of the cylindrical cover (18), this overhang also being formed of an upper planar cut-out (17) positioned obliquely relative to the axis of the cover (18) and of the intermediate support (8), and being terminated by two cut-off faces (19) perpendicular to the axis of the cover.

The nose-shaped overhang (16) is designed to fit into a complementary slot (20) in the head cartridge (see FIGS. 2 to 4).

An end gear (21) of the intermediate support meshes, via the lower cut-out (14) and the upper cut-out (17), with a gear of the head cartridge.

FIG. 5 shows another embodiment of the axial positioning of another head cartridge (22) by another intermediate support (23).

According to this embodiment, the intermediate support (23) is positioned in the stop position on a shoulder (29′) inside the head body and perpendicular to the axis of the support (23), while the cartridge is positioned bearing completely on a shoulder (24) of the head body perpendicular to the axis of rotation of the tool, the underside of the cartridge not comprising a preloaded spring washer as in the preceding embodiment. The relative positioning of the end gear (25) of the support and of the input gear (26) of the head cartridge are guaranteed.

The intermediate support (23) is equipped with a telescopic locking system formed of a sliding lock (27) in the form of a cylindrical sleeve sliding inside a coaxial bushing (28) aligned with the outer body (33) of the support (23).

The sliding lock (27) comprises a projecting internal stop capable of compressing a spring (29) held by a bushing base (30), and comprises an outwardly projecting lug (31) sliding in a guiding slot (32) of the bushing (28) parallel to the longitudinal axis (X) of the support (23).

The lug of the lock can therefore slide axially, but is prevented from rotating.

A compression along the axis (X) of the spring (29) onto the lock (27) generates a component (Z) on the axis of rotation of the head cartridge and ensures an irreversible and effective fit by means of the lug (31) bearing on a plane shoulder (34) of the cartridge, perpendicular to the axis (Z).

The inclination of the lug relative to the axis (X) is given depending on the angle of inclination (α) of the axis (X) relative to the axis (Z).

In the two embodiments, the intermediate support (8) or (23) slides freely in a bore (35) or bores coaxial with the head body (10) until an inner shoulder of said head body (10) is positioned in the axial stop position against an annular shoulder (36) of the body of the intermediate support.

The input gears (37) and end gears (25) or (21) of the intermediate support are conventionally mounted on the same rotation shaft, mounted on bearings inside the outer cylindrical body of the intermediate support.

Other solutions can be conceived of, for example through a protrusion inside the bore in the head body cooperating with a guiding slot (38) provided on the top of the support body and parallel to its axis (X) (see FIG. 4). The intermediate support is rotationally stopped in the head body by the tongue (46) that cooperates with the slot (45) in said head body.

The support body also comprises an extension in the form of a tongue (46) projecting toward the handpiece of the contra-angle, parallel to the axis (X), passing beyond the input gear (37) and having the function explained below.

The head body (10) furthermore has a cylindrical joining part (43) with the handpiece forming a bend with the central part (44) in which the intermediate support slides. For this reason, the cylindrical part (43) comprises an opening (45) in which the tongue (46) is housed, inclined to the central axis of the cylindrical part (43) as it is parallel to the axis (X) of the intermediate support.

The handpiece and its two coaxial subassemblies (3) and (4) will now be described.

The outer subassembly (3) internally supports a reducing assembly (4) that slides freely inside the subassembly (3) but is prevented from rotating, for example by a projecting lug (39) on the inner subassembly (4) and fitting into a slot (40) in the outer subassembly, which is completely uncovered and visible when the casing (5) is fully retracted in the deflector (7).

The inner subassembly comprises a cylindrical overhang (41) passing beyond the output gear (42) of the reducing assembly and comprising a slot (43) and into which the tongue (46) of the intermediate support described above fits.

The reducing assembly comprises a conventional connection (51) to provide the connection with an external motor drive.

The mode of mounting or remounting the various subassemblies will now be described.

On the one hand, the subassemblies of the head body are mounted by introducing the head cartridge (9) into the head body (10) while indexing its position using a pin (11)-groove (12) assembly, then the intermediate support (8 or 23) is introduced either into the internal bore in the head body while indexing the position of the support using the tongue-slot (45, 46) assembly or any other indexing means, and it is pushed until the annular shoulder (36) comes into the stop position against an inner shoulder provided in the head body.

On the other hand, the handpiece is mounted by introducing the inner subassembly (4) into the outer subassembly (3) while indexing them relative to one another by introducing the lug (39) into the slot (40), the casing (5) being retracted, so as to form the handpiece of the contra-angle. Any other indexing means can be envisaged.

The handpiece is then put at the entrance to the head body, while indexing them relative to one another, by introducing the tongue (46) into the clearance (43) so as to ensure the prepositioning approach, the angular connection being guaranteed by the projecting sector of the handpiece (3) and the slot (19) in the head body, until the input gear of the support meshes with the output gear (42) of the inner assembly of the handpiece. Any other indexing means can be envisaged.

In the meshing position, the underside of the tongue (46) is placed against a flat surface (48) provided on the body of the lower subassembly, in the extension of the slot (43).

The mounting-remounting action is completed by moving the casing (5) toward the head body and joining it to the latter, for example by screwing. Through this final action, an inner shoulder of the handpiece exerts an axial thrust on the inner subassembly (4). A slant made on the body (41) redirects this axial thrust onto the oblique support, which locks the cartridge.

Removal starts by disconnecting and retracting the casing (5). The handpiece can now be removed by sliding and the two subassemblies disconnected.

To facilitate removal of the intermediate support, a return (50) projecting outward on the end of the tongue (46) has been provided, along with a clearance (49) in the base of the housing (45) for the tongue, allowing the tip of a tool to be inserted in order to push the return (50) and release the intermediate support.

The device according to the invention is easy to remove and remount, which facilitates adherence to hygiene rules.

The remounting of the various subassemblies is made easier, reliable and quick as all the subassemblies are indexed relative to each other. The indexing means may have alternative embodiments differing from those described by way of nonlimiting example.

The designs of the subassemblies described and claimed are complementary in order to form a new concept of a contra-angle that is completely removable and remountable without using any specific tool, while enabling the transmission of large forces necessary in implantology.

1. A contra-angle for implantology or surgery, the contra-angle comprising:
a head means for holding and rotating an instrument about a longitudinal axis thereof;
a handpiece being comprised of a connection means to an external motor drive, and a reducing assembly with an output gear; and
a plurality of removable subassemblies being two coaxial subassemblies of a removable handpiece and a subassembly called intermediate support, said intermediate support comprising an input gear meshing with said output gear, and an end gear being mounted on a rotating shaft and being housed inside a hollow cylindrical body, said intermediate support being mounted and freely sliding along an axis inside a central part of a head body and enabling positioning in the head and rotational driving of a removable head cartridge, said intermediate support comprising a means for indexing the head body in relation to the handpiece when the subassemblies are mounted together.

2. The contra-angle as claimed in claim 1, wherein the subassemblies comprise means for quick indexing in relation to each other.

3. The contra-angle as claimed in claim 1, wherein said intermediate support is positioned obliquely in the head body, in a direction forming an angle with the direction of the instrument.

4. The contra-angle as claimed in claim 1, further comprising:
one or more flat and axially directed returns of an end cut-out of a cylindrical cover of said intermediate support, being applied on flat bearing surfaces provided in the head cartridge and perpendicular to the axis of rotation of the head, and
a preloaded spring washer being provided between an underside of the head cartridge and a bottom bearing plane of the head body, limiting the residual play between the cartridge and the head body.

5. The contra-angle as claimed in claim 1, wherein said intermediate support is positioned in a stop position on a shoulder inside the head body and perpendicular to the axis of the support, while the cartridge is positioned bearing completely on a shoulder of the head body perpendicular to the axis of rotation of the tool.

6. The contra-angle as claimed in claim 5, wherein said intermediate support is equipped with a telescopic locking system formed of a sliding lock formed of a cylindrical sleeve sliding inside a coaxial bushing aligned with the outer body of the support, the sliding lock comprising a projecting internal stop capable of compressing a spring held by a bushing base, said internal stop comprising an outwardly projecting lug sliding in a guiding slot of the bushing parallel to the longitudinal axis of the support.

7. The contra-angle as claimed in claim 1, wherein a body of said intermediate support also comprises an extension formed of a tongue projecting toward said handpiece, being parallel to the axis, passing beyond the input gear and capable of being housed, when the support is positioned in the head body, in an opening in a cylindrical part of the head body and forming a bend in relation to the central part of the head body.

8. The contra-angle as claimed in claim 7, wherein the handpiece is formed by putting together two subassemblies comprising an inner subassembly supporting a reducing assembly and sliding freely within an outer subassembly, the inner subassembly comprising a cylindrical overhang passing beyond the output gear of the reducing assembly and comprising a slot and into which the tongue of the intermediate support fits.

9. The contra-angle as claimed in claim 1, further comprising:
a central, axially displaceable casing having a withdrawn position, enabling the handpiece and the head body to be disconnected, and
a working position joining the handpiece to the head body.

10. The contra-angle as claimed in claim 9, wherein the casing is joined by being screwed on.

11. The contra-angle as claimed in claim 1, wherein the casing is retracted in the handpiece in a withdrawn position.
